(19) United States
(12) Patent Application Publication
(10) Pub. No.: US 2021/0393324 A1
(43) Pub. Date: Dec. 23, 2021

(54) METHODS FOR CARRYING OUT A CARDIAC PROCEDURE

(71) Applicant: Baylis Medical Company Inc., Montreal (CA)

(72) Inventors: Eduardo Moriyama, Richmond (CA); Matthew DiCicco, Guelph (CA)

(21) Appl. No.: 17/344,211

(22) Filed: Jun. 10, 2021

Related U.S. Application Data
(60) Provisional application No. 63/040,090, filed on Jun. 17, 2020.

(51) Int. Cl. A61B 18/14; A61M 29/02

(52) U.S. Cl. CPC A61B 18/1492; A61B 2017/00247; A61M 29/02

(57) ABSTRACT

A method for carrying out a cardiac procedure includes: a. via an inferior artery, advancing a perforating tip of a perforation device towards an aorta; b. positioning the perforating tip adjacent a wall of the aorta, proximate a left pulmonary artery; and c. advancing the perforating tip to perforate through the wall of the aorta and then through a wall of the left pulmonary artery, to create a pathway between the aorta and the left pulmonary artery.

### FIELD

This document relates to medical procedures. More specifically, this document relates to methods for carrying out a cardiac procedure.

### SUMMARY

The following summary is intended to introduce the reader to various aspects of the detailed description, but not to define or delimit any invention.

Methods for carrying out a cardiac procedure are disclosed. According to some aspects, a method for carrying out a cardiac procedure includes: a. via a superior vein, advancing a radiofrequency perforation electrode of a radiofrequency perforation device towards a superior vena cava; b. positioning the radiofrequency perforation electrode adjacent a wall of the superior vena cava, proximate a right pulmonary artery; and c. delivering radiofrequency energy from the radiofrequency perforation electrode while advancing the radiofrequency perforation device to perforate through the wall of the superior vena cava and then through a wall of the right pulmonary artery, to create a pathway between the superior vena cava and the right pulmonary artery.

In some examples, the method further includes: d. via the superior vein, advancing a dilator over the perforation device to the superior vena cava; and e. after step c., advancing a dilating tip of the dilator over the perforation device and through the pathway to dilate the pathway. The dilator can be a steerable dilator.

In some examples, the method further includes: f. via the superior vein, advancing a sheath over the dilator and the perforation device to the superior vena cava; g. advancing the sheath over the dilator through the pathway; and h. after step g., retracting the dilator through the sheath. The sheath can be a steerable sheath.

In some examples, the method further includes: i. after step g., exchanging the perforation device for a guidewire.

In some examples, the method further includes: i. after step h., delivering a therapeutic device to the pathway via the sheath. Step i. can include positioning a shunt in the pathway or positioning a stent in the pathway.

In some examples, the superior vein is an internal jugular vein.

In some examples, the method further includes: d. advancing a snare towards the right pulmonary artery via a venous access site; and e. after step c., snaring the perforation device with the snare.

In some examples, step d. includes advancing the snare towards the right pulmonary artery via a femoral vein, a hepatic vein, or a superior vein. The method can further include: f. after step e., retracting the snare to advance the radiofrequency perforation electrode out of the body towards the venous access site.

In some examples, the method further includes: delivering a therapeutic device over the radiofrequency perforation device towards the pathway, via the venous access site.

In some examples, at least one of fluoroscopy, angiography, electro-anatomical mapping, intracardiac echocardiography, and transesophageal echocardiography is carried out concurrently with at least one of steps a. to c.

In some examples, the method further includes confirming the creation of the pathway with at least one of fluoroscopy, electro-anatomical mapping, pressure measurement, contrast injection, and echocardiography.

In some examples, the method further includes: advancing a balloon catheter over the perforation device to dilate the pathway.

In some examples, the method further includes: using an anchor system to bring the superior vena cava and the right pulmonary artery together.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example perforation system;

FIG. 2 is a schematic view of a heart, showing a step of an example method for carrying out a cardiac procedure;

FIG. 3 is a schematic view of a heart, showing a step subsequent to that of FIG. 2;

FIG. 4 is a schematic view of a heart, showing a step subsequent to that of FIG. 3;

FIG. 5 is a schematic view of a heart, showing a step subsequent to that of FIG. 4;

FIG. 6 is an enlarged schematic view of a wall of a superior vena cava and a wall of a right pulmonary artery, showing a step subsequent to that of FIG. 5;

FIG. 7 is an enlarged schematic view of a wall of a superior vena cava and a wall of a right pulmonary artery, showing a step subsequent to that of FIG. 6;

FIG. 8 is an enlarged schematic view of a wall of a superior vena cava and a wall of a right pulmonary artery, showing a step subsequent to that of FIG. 7;

FIG. 9 is an enlarged schematic view of a wall of a superior vena cava and a wall of a right pulmonary artery, showing a step subsequent to that of FIG. 8;

FIG. 10 is an enlarged schematic view of a wall of a superior vena cava and a wall of a right pulmonary artery, showing a step subsequent to that of FIG. 9;

FIG. 11 is an enlarged schematic view of a wall of a superior vena cava and a wall of a right pulmonary artery, showing a step subsequent to that of FIG. 10;

FIG. 12 is an enlarged schematic view of a wall of a superior vena cava and a wall of a right pulmonary artery, showing a step subsequent to that of FIG. 13; and

FIG. 13 is an enlarged schematic view of a wall of a superior vena cava and a wall of a right pulmonary artery, showing a step subsequent to that of FIG. 12.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are methods for carrying out cardiac procedures, and more specifically, cardiac procedures in which a pathway (also referred to as a “communication”) is created between the superior vena cava (SVC) and the right pulmonary artery (RPA) of a patient. Such procedures can be carried out, for example, to allow for the insertion of a therapeutic device (e.g. a shunt or a stent) into the pathway, to treat complex univentricular heart or other heart defects.

The methods disclosed herein involve the creation of a pathway between the SVC and the RPA via a superior approach—that is, the SVC can be approached via superior vein (e.g. the internal jugular vein), and a perforation can be created in the wall of the SVC and then into the wall of the RPA. A superior approach can allow for direct access and a less tortuous path to the SVC, length management of the devices used in the procedure, and an ideal force-vector for dilation and placement of an end-therapy device. Furthermore, the methods disclosed herein involve the creation of a pathway using a radiofrequency perforation device. By creating a pathway between the SVC and the RPA using a radiofrequency perforation device, the number of device exchanges can be minimized.

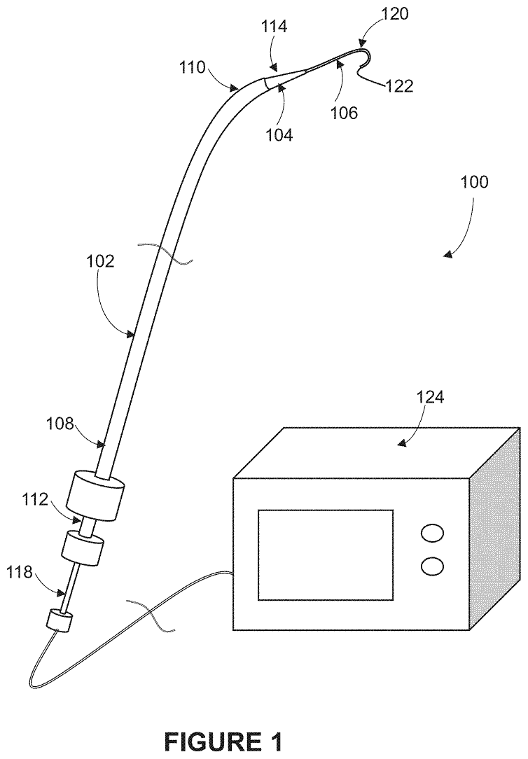

Referring first to FIG. 1, an example system 100 for carrying out a cardiac procedure is shown. In the example shown, the system 100 is a perforation system, and includes a sheath 102, a dilator 104 (the majority of which is within the sheath 102 in FIG. 1), and a radiofrequency (RF) perforation device 106 (the majority of which is within the dilator 104 in FIG. 1).

The sheath 102 can be used to guide various other devices (e.g. the dilator 104, or therapeutic devices such as a stent or shunt) towards a target location in a patient's body (e.g. the SVC). The sheath 102 has a proximal portion 108 and a distal portion 110, and a lumen (not shown) extends through the sheath 102 from the proximal portion 108 to the distal portion 110. The sheath 102 can optionally have a fixed curve, or can be steerable (i.e. the curve can be changed, optionally in more than one plane).

The dilator 104 can be used to dilate a perforation, and has a proximal portion 112 and a distal portion 114 having a dilating tip. A lumen (not shown) extends through the dilator from the proximal portion 112 to the distal portion 114. The dilator 104 can optionally have a fixed curve, or can be steerable (i.e. the curve can be changed, optionally in more than one plane). The dilator 104 can optionally be flexible, to allow it to be compatible with a steerable sheath.

The RF perforation device 106 can be used to perforate a target anatomical structure (e.g. a wall of the SVC), and has a proximal portion 118 and a distal portion 120. The distal portion 120 has a RF perforation electrode 122 at the tip thereof. In the example shown, the distal portion 120 is biased towards a J-shape, to prevent inadvertent perforation of anatomical structures with the RF perforation electrode 122.

The system 100 further includes a RF generator 124, which can be connected to the RF perforation device 106 to deliver RF energy to the RF perforation electrode 122, and to one or more grounding pads (not shown). The RF perforation device 106 can also serve as a support guidewire, as will be described in greater detail below.

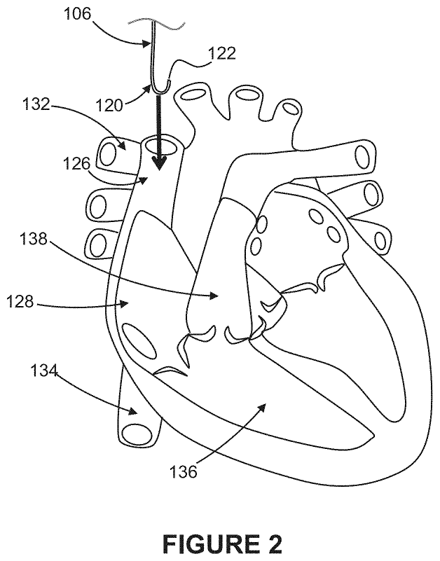

Referring now to FIGS. 2 to 13, an example method for carrying out a cardiac procedure, and specifically for creating a pathway between an SVC and an RPA via a superior vein, will be described. The method will be described with regard to the system 100 shown in FIG. 1; however, the method is not limited to the system 100.

As a first step, a superior vein (e.g. the internal jugular vein, not shown) can be percutaneously accessed (e.g. using a procedure such as a Seldinger technique) and the RF perforation device 106 can be advanced into the superior vein and towards the SVC 126, as shown in FIG. 2. The RF perforation device 106 can be advanced to position the RF perforation electrode 122 in the SVC 126, as shown in FIG. 3.

Optionally, during advancement of the RF perforation electrode 122, the position of the RF perforation electrode 122 can be confirmed using fluoroscopy (e.g. in examples wherein the RF perforation device 106 includes one or more radiopaque markers or features), angiography, electro-anatomical mapping (EAM) (e.g. to confirm real-time positioning of the RF perforation electrode 122 using real-time or pre-determined computerized tomography data, in conjunction with a catheter or guidewire with one or more EAM markers in the right atrium 128), intracardiac and/or transesophageal echocardiography (ICE and/or TEE) (e.g. using echogenic markers or features on the RF perforation device 106).

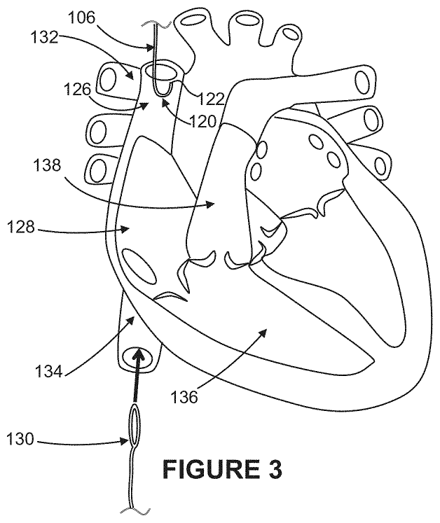

Referring to FIG. 3, as a next step (or earlier or later in the method, for example prior to the previous steps or after the step shown in FIG. 8), a snare 130 can be advanced percutaneously towards the RPA 132. The snare 130 can be advanced, for example, via a venous access site (not shown) such as the femoral vein, the hepatic vein, or a superior vein. Referring to FIG. 4, in the example shown, the snare 130 is advanced via the femoral vein (not shown), the inferior vena cava 134, the right atrium 128, the right ventricle 136, and the pulmonary trunk 138, until the snare 130 reaches the RPA 132. In FIG. 4, the portion of the snare 130 that is within the RPA 132 is shown in dotted line, as the RPA 132 is behind the aorta and the SVC 126.

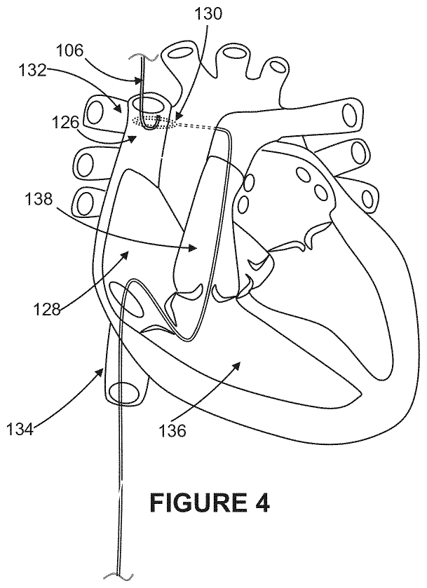

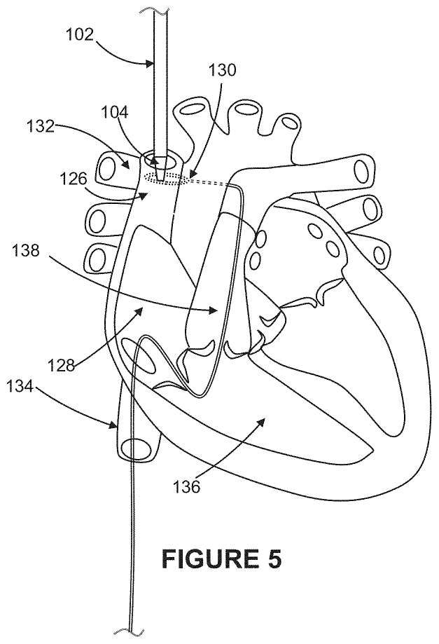

Referring to FIG. 5, as a next step, the dilator 104 and the sheath 102 may be advanced towards the SVC 126 via the superior vein. The dilator 104 and sheath 102 can be advanced together over the perforation device 106 (not visible in FIG. 5), with the dilator 104 received in the sheath 102, or can be advanced in sequence, e.g. by advancing the dilator 104 over the perforation device 106 and then advancing the sheath 102 over the dilator 104. The dilator 104 and sheath 102 can be advanced until the dilating tip of the dilator 104 is flush with the RF perforation electrode 122 (not visible in FIG. 5) of the RF perforation device 106. In alternative examples, a large steerable dilator (not shown) can replace the sheath 102 and dilator 104.

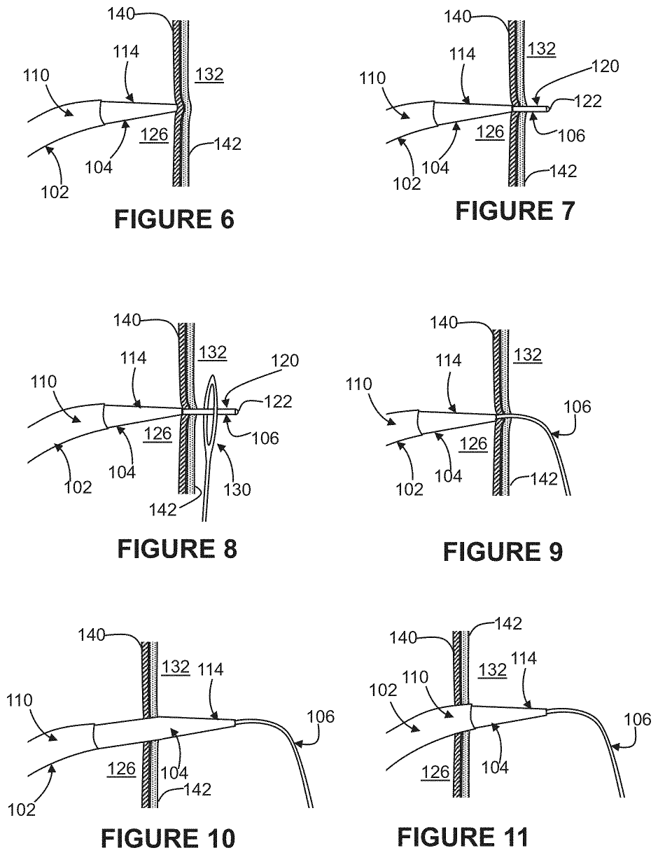

With the distal portion 110 of the sheath 102, distal portion 114 of the dilator 104, and distal portion 120 of the RF perforation device 106 in the SVC 126, the sheath 102 and dilator 104 can be maneuvered to direct the RF perforation electrode 122 towards a desired perforation site—i.e. the wall 140 of the SVC 126, proximate a wall 142 of the RPA 132, as shown in FIG. 6. This can be achieved by steering the sheath 102 and/or dilator 104 (in examples wherein the sheath 102 and/or the dilator 102 are steerable), or by adjusting the position of the sheath 102 and/or dilator 104. This step can optionally be facilitated using fluoroscopy (e.g. in examples wherein the RF perforation device 106 includes one or more radiopaque markers or features), angiography, electro-anatomical mapping (EAM) (e.g. to confirm real-time positioning of the RF perforation electrode 122 using real-time or pre-determined computerized tomography data, in conjunction with a catheter or guidewire with one or more EAM markers in the right atrium 128, intracardiac and/or transesophageal echocardiography (ICE and/or TEE) (e.g. using echogenic markers or features on the RF perforation device 106).

Referring to FIG. 7, the RF perforation electrode 122 can then be energized with RF energy, and the RF perforation device 106 can be advanced to perforate through the wall 140 of the SVC 126 and through the wall 142 of the RPA 132 to create a pathway between the SVC 126 and the RPA 132. Optionally, creation of the pathway can be confirmed using fluoroscopy, electro-anatomical mapping, pressure measurement, contrast injection, and intracardiac and/or transesophageal echocardiography.

Referring to FIGS. 8 and 9, the snare 130 can then be used to capture the distal portion 120 of the RF perforation device 106, and the snare 130 can then be retracted to advance the RF perforation device 106 towards the venous access site (e.g. the femoral vein, not visible in FIGS. 8 and 9), optionally to externalize the RF perforation electrode 122. Optionally, the RF perforation device 106 can be “flossed” to enlarge the pathway.

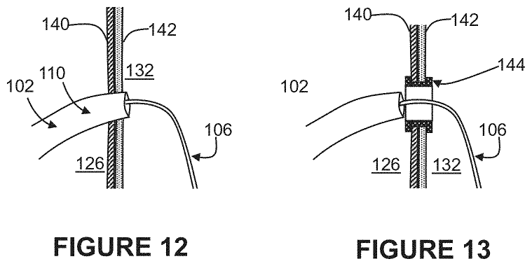

Referring to FIG. 10, the dilating tip of the dilator 104 can then be advanced over the RF perforation device 106 and through the pathway, to dilate the pathway. Referring to FIG. 11, the sheath 102 can then be advanced over the dilator 104 and through the pathway. Referring to FIG. 12, the dilator 104 (not visible in FIG. 12) can then be retracted back towards the superior vein (not shown) and removed from the body, leaving the sheath 102 and the RF perforation device 106 in place in the pathway.

Optionally, after perforation, the RF perforation device 106 can be exchanged for another wire (either via the arterial access site or the venous access site), such as a relatively stiff guidewire.

Optionally, after perforation, an anchor device such as a balloon can be advanced via the sheath 102 and used to bring the SVC 126 and the RPA 132 together.

Optionally, after perforation, a balloon catheter can be advanced over the RF perforation device 106 and via the sheath 102 to enlarge the pathway.

Referring to FIG. 13, with the sheath 102 and the RF perforation device 106 (or another guidewire) in place in the pathway, a therapeutic device can be delivered to the pathway via the sheath 102, over the RF perforation device 106. The therapeutic device can be for, example, a shunt (e.g. shunt 144, shown in FIG. 13) or a stent that is positioned in the pathway.

Alternatively, the sheath 102 can be retracted back towards the superior vein, and a secondary sheath—e.g. a large bore sheath designed for therapeutic device delivery—can be advanced over the perforation device 106 (or another guidewire) via the superior vein. The secondary sheath can then be used to deliver a therapeutic device to the pathway.

Alternatively, delivery of the therapeutic device can be achieved by delivering the therapeutic device over the RF perforation device 106 via the venous access site (e.g. the femoral vein). For example, a secondary sheath (e.g. a large bore sheath, not shown) can be advanced over the RF perforation device 106 via the venous access site, to the right pulmonary artery. The secondary sheath can then be used to deliver the therapeutic device.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for carrying out a cardiac procedure, comprising:
a. via a superior vein, advancing a radiofrequency perforation electrode of a radiofrequency perforation device towards a superior vena cava;
b. positioning the radiofrequency perforation electrode adjacent a wall of the superior vena cava, proximate a right pulmonary artery; and
c. delivering radiofrequency energy from the radiofrequency perforation electrode while advancing the radiofrequency perforation device to perforate through the wall of the superior vena cava and then through a wall of the right pulmonary artery, to create a pathway between the superior vena cava and the right pulmonary artery.

2. The method of claim 1, further comprising:
d. via the superior vein, advancing a dilator over the perforation device to the superior vena cava; and
e. after step c., advancing a dilating tip of the dilator over the perforation device and through the pathway to dilate the pathway.

3. The method of claim 2, wherein the dilator is a steerable dilator.

4. The method of claim 2, further comprising:
f. via the superior vein, advancing a sheath over the dilator and the perforation device to the superior vena cava;
g. advancing the sheath over the dilator through the pathway; and
h. after step g., retracting the dilator through the sheath.

5. The method of claim 4, further comprising:
i. after step g., exchanging the perforation device for a guidewire.

6. The method of claim 4, further comprising:
i. after step h., delivering a therapeutic device to the pathway via the sheath.

7. The method of claim 6, wherein step i. comprises positioning a shunt in the pathway.

8. The method of claim 6, wherein step i. comprises positioning a stent in the pathway.

9. The method of claim 4, wherein the sheath is a steerable sheath.

10. The method of claim 1, wherein the superior vein is an internal jugular vein.

11. The method of claim 1, further comprising:
d. advancing a snare towards the right pulmonary artery via a venous access site; and
e. after step c., snaring the perforation device with the snare.

12. The method of claim 11, wherein step d. comprises advancing the snare towards the right pulmonary artery via a femoral vein, a hepatic vein, or a superior vein.

13. The method of claim 11, further comprising:
f. after step e., retracting the snare to advance the radiofrequency perforation electrode out of the body towards the venous access site.

14. The method of claim 13, further comprising delivering a therapeutic device over the radiofrequency perforation device towards the pathway, via the venous access site.

15. The method of claim 1, wherein at least one of fluoroscopy, angiography, electro-anatomical mapping, intracardiac echocardiography, and transesophageal echocardiography is carried out concurrently with at least one of steps a. to c.

16. The method of claim 1, further comprising confirming the creation of the pathway with at least one of fluoroscopy, electro-anatomical mapping, pressure measurement, contrast injection, and echocardiography.

17. The method of claim 1, further comprising advancing a balloon catheter over the perforation device to dilate the pathway.

18. The method of claim 1, further comprising using an anchor system to bring the superior vena cava and the right pulmonary artery together.
